(19) United States
(12) Patent Application Publication
(10) Pub. No.: US 2024/0226065 A1
(43) Pub. Date: Jul. 11, 2024

(54) USE OF RAGE INHIBITORS TO TREAT CANCER-RELATED COGNITIVE DECLINE

(71) Applicant: Georgetown University, Washington, DC (US)

(72) Inventors: Marc E. Lippman, Washington, DC (US); Barry I. Hudson, Falls Church, VA (US); Jeanne Mandelblatt, Garrett Park, MD (US); G. William Rebeck, Washington, DC (US)

(73) Assignee: Georgetown University, Washington, DC (US)

(21) Appl. No.: 18/290,252

(22) PCT Filed: May 11, 2022
(86) PCT No.: PCT/US2022/028741

§ 371 Date: Nov. 10, 2023

Related U.S. Application Data
(60) Provisional application No. 63/187,351, filed on May 11, 2021.

(51) Int. Cl. A61K 31/4164; A61K 31/166; A61K 45/06; A61P 25/28

(52) U.S. Cl. CPC A61K 31/4164; A61K 31/166; A61K 45/06; A61P 25/28

(57) ABSTRACT

A method of treating cancer-related cognitive decline (CRCD) in a patient, in which the method comprises administering to the patient an effective amount of an inhibitor of receptor for advanced glycation endproducts (RAGE). The inhibitor of RAGE may be administered as a single dose or as multiple doses before the administration of the cancer therapy, during the administration of the cancer therapy, after the administration of the cancer therapy, or a combination thereof. Administration of the inhibitor of RAGE may improve attention, processing speed, executive functioning, learning, memory, or a combination thereof, of the patient as compared to patients who are not administered the inhibitor of RAGE.

### CROSS-REFERENCE TO RELATED APPLICATIONS

This application claims the benefit of U.S. Provisional Application No. 63/187,351, filed on May i1, 2021, which is incorporated herein by reference in its entirety.

### STATEMENT REGARDING FEDERALLY SPONSORED RESEARCH OR DEVELOPMENT

This invention was made with government support under grant number R01 AG067258 awarded by the National Institutes of Health. The government has certain rights in the invention.

### FIELD OF INVENTION

The present invention generally relates to methods and treatments of cognitive decline or impairment. In particular, the present invention relates to the use of inhibitors of receptor for advance glycation endproducts (RAGE) to treat or prevent cognitive decline or impairment induced by cancer therapy.

### BACKGROUND OF THE INVENTION

The receptor for advanced glycation end-products (RAGE) is a transmembrane protein which belongs to the immunoglobulin superfamily of receptors (Neeper et a1., 1992) The ligands for RAGE include advanced glycation end-products (AGEs), as well as non-AGE ligands such as amyloid β, S100s, nucleic acids (RNA, DNA), high mobility group box 1, beta-sheet fibrils, Mac-1, and collagen I/IV (Hudson and Lippman, 2018). RAGE signaling is up regulated during chronic and sustained inflammatory conditions, and has been linked to the development and progression of diabetic vascular complications, cardiovascular disease, cancer, Alzheimer's disease, and other inflammatory diseases (Hudson and Lippman, 2018; Kalea et al, 2009; Kierdorf and Fritz, 2013).

Cancer-related cognitive decline (CRCD) is a known occurrence in patients who have cancer and/or are receiving cancer therapy. It can manifest across various domains of cognition, including working memory, attention, concentration, processing speed, and executive functioning (Anderson-Hanley et al., 2003; Janelsins et al, 2014). CRCD is most common during and immediately after cancer therapy, with most individuals showing recovery over time. Some individuals have a persistent, but stable cognitive impairment, and a small number show increasing decline over time (Ahles et al., 2012).

CRCD has been reported since the 1970s (Silberfarb, 1983). Historically, cognitive changes in cancer patients were assumed to be psychological-related, associated with factors such as depression or anxiety, or were due to other side effects of cancer treatments like fatigue. (Ahles and Saykin, 2007). Such an assumption has been dismissed, due to studies that found that the cognitive changes persisted post-chemotherapy after statistically controlling for psychological factors or fatigue, and that the cognitive changes occurred in patients without significant psychological distress or fatigue (Ahles and Saykin, 2007).

The mechanisms by which CRCD occur remain unknown. Researchers have studied the blood-brain barrier, DNA damage and oxidative stress, cytokine deregulation, genetic susceptibility, and estrogen or testosterone reduction for their impact on changing cognitive functioning (Ahles and Saykin, 2007), but the etiology of CRCD remains elusive (Ahles and Saykin, 2007; Mandelblatt et al, 2014).

With the exception of therapies tested in patients with brain cancer, there are no pharmacological treatments available for CRCD. This change in cognitive function differs from Alzheimer's disease and other neurodegenerative diseases because it is not deterministically progressive nor is it associated with pathological brain changes like plaques and amyloid.

Cognitive deficits can greatly affect survivorship due to problems with daily function, compliance with treatment and surveillance follow-up (Lange et al, 2014; Bluethmann et at, 2017). Thus, there remains a high unmet need in the field for an effective means of treating and/or preventing CRCD.

### SUMMARY OF INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The invention provides methods and compositions for patients with CRCD.

In one aspect., the invention provides methods of treating CRCD in a patient in need thereof, the method comprising administering to the patient an effective amount of an inhibitor of RAGE. In another aspect, the invention provides methods of preventing CRCD in a patient in need thereof, the method comprising administering to the patient an effective amount of an inhibitor of RAGE. In yet another aspect, the invention provides methods of alleviating symptoms of CRCD in a patient in need thereof, the method comprising administering to the patient an effective amount of an inhibitor of RAGE.

In one aspect, the invention provides pharmaceutical compositions comprising an effective amount of a RAGE inhibitor for use in treating CRCD in a patient in need thereof. In another aspect, the invention provides pharmaceutical compositions comprising an effective amount of a RAGE inhibitor for use in preventing CRCD in a patient in need thereof. In a further aspect, the invention provides pharmaceutical compositions comprising an effective amount of a RAGE inhibitor for use in alleviating symptoms of CRCD in a patient in need thereof.

In some embodiments, the CRCD is induced by a hormonal cancer therapy, a chemotherapy, or a targeted therapy. In preferred embodiments, the CRCD is induced by a chemotherapy.

In some embodiments, the patient comprises an apoliprotein E4 (APOE4) genotype.

In some embodiments, the inhibitor of RAGE is administered as a single dose before the administration of the cancer therapy, during the administration of the cancer therapy, or after the administration of the cancer therapy. In other embodiments, the inhibitor of RAGE is administered as multiple doses for a duration of time. The duration of time may be before the administration of the cancer therapy, during the administration of the cancer therapy, after the administration of the cancer therapy, or a combination thereof.

The administration of the inhibitor of RAGE may improve attention, processing speed, executive functioning, learning, memory, visuospatial ability, or a combination thereof, of the patient as compared to patients who are not administered the inhibitor of RAGE. The administration of the inhibitor of RAGE may prevent reduction in attention, processing speed, executive functioning, learning, memory, visuospatial ability, or a combination thereof, of the patient. Further, the administration of the inhibitor of RAGE may alleviate symptoms of CRCD demonstrated by a reduction in attention, processing speed, executive functioning, learning, memory, visuospatial ability, or a combination thereof, of the patient.

### BRIEF DESCRIPTION OF THE DRAWING FIGURES

FIG. 1 shows results from a Barnes maze, as described in Example 1. The Barnes maze was used to assess cognition in mice that were administered a vehicle control (Group 1, n=8); doxorubicin, which is a cancer chemotherapy agent (Group 2, n=8); or doxorubicin and FPS-ZM1, which is a RAGE inhibitor (Group 3, n=8). FIG. 1 shows for each of the groups the time to escape the maze (i.e., latency) as an average of four trials (mean +/− standard error) on the final day that the mice were trained.

### DETAILED DESCRIPTION OF THE INVENTION

The practice of the present invention can employ, unless otherwise indicated, conventional techniques of oncology, pharmaceutics, formulation science, protein chemistry, cell biology, molecular biology, recombinant DNA, immunology, pre-clinical pharmacology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and 13, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their System International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth.

Likewise, a disclosed range is a disclosure of each individual value encompassed by the range. For example, a stated range of 5-10 is also a disclosure of 5, 6, 7, 8, 9, and 10.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of an active agent is an amount sufficient to carry out a specifically stated purpose.

The term “pharmaceutical composition” refers to a preparation that is in such form as to permit the biological activity of the active ingredient to be effective and which contains no additional components that are unacceptably toxic to a subject to which the composition would be administered.

As used herein, the terms “treating” and “treatment” refer to reduction in severity and/or frequency of symptoms, elimination of symptoms and/or underlying cause, and/or improvement or remediation of damage.

As used herein, the terms “prevent” and “preventing” refer to stopping the occurrence of symptoms and/or their underlying cause.

As used herein, the terms “alleviate” and “alleviating” refer to any decrease or lessening in occurrence or activity or severity of a symptom.

### RAGE Inhibitors

As used herein, an inhibitor of RAGE, or “RAGE inhibitor”, may be any agent that can reduce the activity, function, or expression of RAGE. The activity, function or expression may be completely suppressed, i.e., no activity, function or expression, or the activity, function or expression may simply be lower in with exposure to the RAGE inhibitor as compared to no exposure to the RAGE inhibitor.

In some embodiments, the RAGE inhibitor may block the upstream pathway of RAGE such that RAGE is not activated or its activity is reduced. In some embodiments, the RAGE inhibitor may block the activity, function, or expression of downstream effector molecules of activated RAGE such that RAGE cannot propagate any signal or can only propagate a reduced signal.

In some embodiments, the RAGE inhibitor is a small molecule compound. Examples of such small molecule compounds may include, but are not limited to, azeliragon (3-[4-[2-butyl-1[4-(4-chlorophenoxy) phenyl]imidazol-4-yl]phenoxy]-N,N-diethylpropan-1-amine; also known TTP488), derivatives of azeliragon (see Lee et al., 2012), FPS1, FPS2, FPS3, FPS-ZM1 (Deane et al., 2012), 4,6-bis(4-chlorophenyl)pyrimidine analog 59 (Han et al., 2012), 4-fluorophenoxy analog 40 (Han et at., 2014), 3-(N,N-dimethylamino)pyrrolidine analog I2o (Han et al., 2015), and RAGE inhibitors disclosed in U.S. Patent Application Publication No. 2008/0119512, U.S. Patent Application Publication No. 2010/0254983, U.S. Pat. No. 7,361,678, PCT International Application Publication No. WO 2007/089616, PCT International Application Publication No. WO 2007/076200, and PCT International Application Publication No. WO 2007/0286858, all of which are hereby incorporated by reference.

In some embodiment, RAGE inhibition may be through the use of RNA interference (RNAi). RNAi techniques are well known and rely of double-stranded RNA (dsRNA), where one stand of the dsRNA corresponds to the coding strand of the mRNA that codes for RAGE, and the other strand is complementary to the first strand. The requirements of optimal RNAi species for a given nucleotide sequence are well-known or can be readily ascertained given the state of the art. For example, it is known that optimal dsRNA is about 20-25nt in length, with a 2 base overhand on the 3 end of each strand of the dsRNA, often referred to as short interfering RNAs (siRNA). Other well-known configurations such as short hairpin RNA (shRNA) may also work. shRNAs are one continuous RNA strand where a portion is self-complementary such that the molecule is double-stranded in at least one portion. It is believed that the cell processed shRNA into siRNA. The term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand. Accordingly, some embodiments of the invention involve a RAGE inhibitor comprising at least one RNAi molecule and/or at least one antisense molecule. In certain embodiments, the RNAi molecule and/or antisense molecule is specific towards RAGE. The RNAi molecules and/or antisense molecules may also be part of a complex, such as a liposomal complex that can be used to deliver the RNAi molecules or antisense/molecules.

In some embodiments, the RAGE inhibitor may comprise DNA expression vectors that encode the RNTAi molecules and/or antisense molecules. Certain embodiments can utilize only one vector, for example when the RNAi molecule is a shRNA, or when opposing promoters are placed on either side there of the coding sequence for the RNAi molecule.

Thus, a RAGE inhibitor includes the use of DNA that, when transcribed, can block the activity, function, or production of RAGE. The liposomal delivery systems described above are one way in which the DNA encoding an RNAi and/or antisense can be delivered to the target.

Alternatively, the DNA encoding an RNAi and/or antisense can be prepared in a viral vector system that has the capability of entering into cells. These are well-known in the art and include papovavirus SV40, adenovirus, vaccinia virus, adeno-associated virus, herpes simplex virus, Epstein-Barr virus, retrovirus, and baculovirus.

The RAGE inhibitor may be formulated in a composition, e.g., a pharmaceutical composition. Therefore, some aspects of the present invention relates to a composition comprising a RAGE inhibitor. Preferably, the composition comprises one or more carriers, diluents, excipients, or other additives. For example, the composition can comprise one or more bulking agents (e.g., dextran 40, glycine, lactose, mannitol, trehalose), one or more buffers (e.g., acetate, citrate, histidine, lactate, phosphate, Tris), one or more pH adjusting agents (e.g., hydrochloric acid, acetic acid, nitric acid, potassium hydroxide, sodium hydroxide), and/or one or more diluents (e.g., water, physiological saline). The pH of the composition is preferably between about 3.0 and 8.0. In some embodiments, the pH is between about 3.5 and 6.5, or between about 5.0 and 7.5.

### Methods of Use of RAGE Inhibitors

The present invention relates to the use of a RAGE inhibitor in patients who are undergoing cancer therapy and may, or are, experiencing CRCD. Therefore, in some aspects, the present invention relates to: (i) a method of treating CRCD in a patient in need thereof; (ii) a method of preventing CRCD in a patient in need thereof; or (iii) a method of alleviating symptoms of CRCD in a patient in need thereof. These methods may comprise administering an effective amount of a RAGE inhibitor to the patient.

In some aspects, the present invention relates to the use of a RAGE inhibitor for (i) treating CRCD in a patient in need thereof; (ii) preventing CRCD in a patient in need thereof; or (iii) alleviating symptoms of CRCD in a patient in need thereof. These uses may comprise administering an effective amount of a RAGE inhibitor to the patient.

In some aspects, the present invention relates to a RAGE inhibitor for use in (i) treating CRCD in a patient in need thereof; (ii) preventing CRCD in a patient in need thereof; or (iii) alleviating symptoms of CRCD in a patient in need thereof. These uses may comprise administering an effective amount of a RAGE inhibitor to the patient.

The cancer therapy may be a hormonal cancer therapy, a chemotherapy, or a targeted therapy (e.g., herceptin). In preferred embodiments, the cancer therapy is chemotherapy.

The chemotherapy may comprise, for example, alkylating agents, antimetabolites, anti-microtubule agents, topoisomerase inhibitors, or cytotoxic antibiotics. Examples of chemotherapy may include, but are not limited to, 5-fluorouracil, 6-mercaptopurine, actinomycin-D, bleomycin, capecitabine, carboplatin, cisplatin, cyclophosphamide, cytarabine, dacarbazine, daunorubicin, docetaxel, doxorubicin, epirubicin, estramustine, etoposide, fludarabine, folinic acid, gemcitabine, irinotecan, melphalan, methotrexate, mustine, oxaliplatin, paclitaxel, pemnetrexed, prednisolone, procarbazine, temozolomide, teniposide, topotecan, vinblastine, vincristine, and vinorelbine.

The patient may have any cancer for which a therapy, preferably a chemotherapy, is required or recommended to treat. Examples of cancers include, but are not limited to, bladder, breast, cervical, colorectal, germ cell, intestinal, leukemia, lung, lymphoma (Hodgkin's or non-Hodgkin's), multiple myeloma, ovarian, sarcoma, and stomach.

In some embodiments, the patient may comprise the APOE4 genotype, which has been determined to be a genetic risk factor for CRCD (Ahles et al., 2003, Mandelblatt et al., 2018, Demby et al., 2020). The relationship between the APOE4 genotype and CRCD has been demonstrated in preclinical studies in APOE4 knock-in mice (Speidell et at, 2019; Demby et al, 2020), as well as in clinical studies of APOE4+ breast cancer patients receiving chemotherapy (Mandelblatt et al, 2018).

The RAGE inhibitor may be administered to a patient at a dose of about 0.1 to 500 mg/kg, or about 0.5 to 400 mg/kg, or about 0.5 to 300 mg/kg, or about 0.5 to 200 mg/kg, or about 0.5 to 100 mg/kg, including, for example, a dose of about 0.5, or about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 80, or about 85, or about 90, or about 95, or about 100 mg/kg.

The RAGE inhibitor may be administered to a patient at a dose of about 1 to 1000 mg, or about 5 to 900 mg, or about 5 to 800 mg, or about 5 to 700 mg, or about 5 to 600 mg, or about 5 to 500 mg, including, for example, a dose of about 5, or about 10, or about 15, or about 20, or about 25, or about 30, or about 35, or about 40, or about 45, or about 50, or about 60, or about 70, or about 80, or about 90, or about 100, or about 120, or about 130, or about 140, or about 150, or about 160, or about 170, or about 180, or about 190, or about 200, or about 220, or about 240, or about 260, or about 280, or about 300, or about 320, or about 340, or about 360, or about 380, or about 400, or about 420, or about 440, or about 460, or about 480, or about 500 mg.

The RAGE inhibitor may be administered parenterally, orally, or topically. Parenteral routes of administration include intravenous (IV), intramuscular, intraperitoneal, intrathecal, and subcutaneous.

The RAGE inhibitor may be administered as a single dose or as multiple doses over a duration of time, such that each dose is administered periodically, for example, daily, every two days, every three days, every four days, every five days, every six days, weekly, every two weeks, every three weeks, every four weeks, monthly, every two months, every three months, every four months, every five months, every six months, or any duration therebetween. In embodiments in which the RAGE inhibitor is administered as a single dose, the administration may occur before, during, or after the administration of the cancer therapy. In embodiments in which the RAGE inhibitor is administered as multiple doses over a duration of time, the duration of time may be before, during, after, or a combination thereof (e.g., overlapping both before and during; overlapping both during and after; overlapping before, during, and after; administered before and after).

Efficacy of treatment, prevention, or alleviation of symptoms of CRCD can be evaluated by one or more known measures. For example, in some embodiments, a patient subjected to methods of the invention may be evaluated by comparing cognition decline or impairment to baseline, i.e., before the patient received the cancer therapy. A baseline assessment is preferably performed within 24, 48, or 72 hours, or within 1, 2, 3, or 4 weeks prior to the first administration of the RAGE inhibitor.

In some embodiments, a patient subjected to methods of the invention may be evaluated by comparing cognitive decline or impairment to patients receiving the same cancer therapy and/or having the same type of cancer who are not subjected to the methods of the invention, i.e., control patients. Cognitive decline or impairment in a patient treated by a method of the invention can be compared, for example, to the median cognitive decline or impairment in a population of control patients. The population of control patients can be administered, for example, a placebo. Comparisons can be analyzed statistically using, for example, the Wilcoxon signed rank test.

Cognitive decline or impairment may be assessed through neuropsychological testing or self-reporting. The testing may include one or more of FACT-Cog PCI subscale; neuropsychological tests of attention, processing speed, executive function, learning, language, memory, and/or visuospatial ability; or a combination thereof.

The neuropsychological tests of attention, processing speed, executive function, learning, language, memory, and visuospatial ability may be tests that are known in the art.

Examples of such tests include, but are not limited to:
Oral Trailmaking—Part A. This is a neuropsychological measure that provides an assessment of sequential set-shifting without the motor and visual demands of the written TMT. Part A requires a subject to count from 1-25 as quickly as he/she is able.
Oral Trailmaking—Part B. This is a neuropsychological measure that provides an assessment of sequential set-shifting without the motor and visual demands of the written TMT. Part B requires a subject to alternate from numbers to letters from 1-A to 13-M.
Symbol Subtest-Wechsler Adult Intelligence Test-ill (D 1997). This test measures visual-motor coordination, psychomotor speed, visual attention, and incidental learning. Adequate performance on the test requires intact functioning across several domains, so it is very sensitive to impairment.
Symbol Digit Modalities Test. This test detects cognitive impairment by looking at visual attention and incidental learning. The test involves seeing the digit-symbol key and orally telling the examiner the correct number that is paired with a random symbol.
Controlled Oral Word Association Test (COW AT), A 2006). This test is a measure of verbal fluency, cognitive flexibility, and semantic knowledge. Participants are given three one-minute trials to generate words as quickly as possible to each of three letter cues (F, A, and S). The test is scored based on the total number of words generated.
Mental Control Subtest-Weschler Memory Scale. This subtest of the WMSIII assesses speeded performance for reciting the alphabet, counting forward and backward, days of the week forward and backward, months of the year forward and backward, counting by 6s while stating days of the week in order.
Driving Scenes Test from the NAB. This test of working memory, selective attention, and visual scanning is a Daily Living test with excellent ecological validity (Brown, et al., 2005). Participants are shown a color line drawing of a road scene (from the driver's perspective) each for a 30-second exposure and then shown another scene and asked to report new and missing items from the previous scene; this is repeated 6 times. Scores range from 0 (worst) to 70 (best).
The Timed Instrumental Activities of Daily Living (TLADL). The TIADL is a standardized timed assessment of performance of five instrumental activities of daily living: finding a telephone number in a phone directory, finding, and counting out correct change, finding and reading out the first three ingredients on a food can, finding two specific food items on a shelf of food, and finding and reading the directions on a medicine container. (Owsley, et at., 2002). Each task has a preset maximum time duration after which the task is terminated. Scoring is based on a combination of the completion time and an error code for each task.
Figure Drawing from the NAB—Organization Subscale. This subscale of the test is designed to measure fragmentation, planning, and overall organizational skill. The complex figure is presented, and the participant is instructed to copy the figure on a separate piece of paper. The examiner switches the color pen the participant is using in order to record the order in which the figure was drawn. The forms will be alternated for each visit, with Form 1 administered at baseline, Form 2 at 12-month follow-up, and Form 1 at 24-month follow-up. Planning and fragmentation of the lines are together scored to give an overall organization score.
Boston Naming Test (BNT). The BNT is the most commonly used test of confrontation naming (i.e., lexical retrieval) and has been found to be sensitive to minor or early aphasic deficits. (Kaplan, et al, 1983). An age, education, and gender-corrected standard score, based on the number correct, can be calculated.
Verbal Naming. The measure is a nonvisual measure of word finding with stimuli chosen based on rare frequency of usage in spoken English. Fifty-five objects or verbs are described, and subjects must name the object or verb. Subjects are given a phonemic cue if they are unable to find the correct word.
Category Fluency Test. This test is a measure of verbal fluency and ability to access semantic knowledge. Participants are given one minute to name as many words as possible that belong to a particular semantic category (e.g., animals). The overall score is the number of words correctly generated (Benton, 1994).
NAB Digits Forward. This test is a version of the commonly used digit span paradigm, which evaluates auditory attentional capacity. The NAB test involves seven items, each with two trials, in which the examinee is asked to repeat series of digits orally, with spans ranging from 3 to 9 digits.
NAB Digits Backward. This test is considered a measure of attention and to a lesser extent, working memory (Lezak, el al., 2004). It involves seven items, each with two trials, in which the examinee is asked to repeat series of digits orally in reverse order.
Brief Test of Attention. This is a commonly used neuropsychological measure of auditory-divided attention that was developed to reduce the influence of confounding task demands such as motor speed and visual scanning
Logical Memory I and II, Wechsler Memory Scale (WMS-IV). (D 1997, D 2008).

This test involves the examinee listening to a brief paragraph/story and then immediately freely recalling as much as remembered. Following a 30-minute delay, the examinee is asked to recall as much as possible from one story only (IA and IIA). In some embodiments, the Scaled Score for Logical Memory I1 may be used in the summary score.
List Learning from the NAB. This is a 12-word list learning task with three learning trials, followed by an interference list, and then a short delay free recall. The word list includes three embedded semantic categories with four words in each category. Following a 10-15 minute delay, there is a free recall of the initial list, followed by a 36-item forced-choice recognition task. In some embodiments, two alternative forms of the NAB list may be used. In such embodiments, the forms may be alternated for each visit with Form I being administered at baseline, Form 2 at 12-month follow-up, and Form 1 at 24-month follow-up. Scores resulting from this test include measures of sensitivity to interference, the use of semantic encoding as an organizational mnemonic strategy, delayed free recall, improvement from free recall to recognition, and discrimination. Visual-spatial functioning may be evaluated through different tests known in the art. Examples of such tests include, but are not limited to:
Figure Drawing from the NAB—Copy Subscale. This subscale of the test is designed to measure visuospatial and visuoconstruction skills. A complex figure is presented, and the participant is instructed to copy the figure on a separate piece of paper. The examiner switches the color pen the participant is using in order to record the order in which the figure was drawn. The forms will be alternated for each visit. Form 1 will be administered at baseline, Form 2 at 12-month follow-up, and Form 1 at 24-month follow-up. The primary score used in this visuospatial domain is based on the ability to reproduce the figure correctly/accurately, distinct from the organizational aspects of the production.

Symptoms of CRCD may comprise one or more problems with memory, attention, processing speed, learning, concentration, and/or executive functioning. These symptoms may be based on observational evidence by a trained physician in the field, or may be based on neurological testing as described herein.

### EXAMPLES

### Example 1

A study was conducted to evaluate the effects of RAGE inhibition on preventing CRCD. The study was performed using a mouse model of C57BL/6 J mice having the human APOE4 allele in the mouse APOE gene locus.

### Method

In the study, 24 female APOE mice were divided evenly into three treatment groups: Group 1, which received a vehicle control; Group 2, which received doxorubicin, a chemotherapy medication used to treat a variety of cancers that include breast cancer, bladder cancer, Kaposi's sarcoma, lymphoma, and acute lymphocytic leukemia; and Group 3, which received doxorubicin and FPS-ZM1, RAGE antagonist. Groups 2 and 3 were administered the doxorubicin as two doses of 5 mg/kg, separated by one week. Group 3 was administered 2 mg/kg of FPS-ZMI twice per week by intraperitoneal injection starting one week before the first administration of doxorubicin and continuing throughout the study.

Four weeks after doxorubicin treatment (or the vehicle control) was completed, mice were analyzed for hippocampal based memory via the Barnes maze (San Diego Instruments Inc., San Diego, CA). The Barnes maze is used in mice as a behavioral measure of animal cognition, and can assess spatial learning and memory. The maze was in a space containing extra-maze cues, illumination of 150 lx and 75 dB white noise; it had 19 shallow decoy containers and one escape hole around the edge of the disk. The apparatus was captured by overhead video and assessed using ANY-maze™ software.

The maze analysis consisted of one habituation day followed by four consecutive training days. On the habituation day, a habituation task was conducted that consisted of a single trial with the escape hole placed in a location that would not be used for the remainder of the analysis. On the training days, the mice were put through four trials separated by an interval of 15 minutes, and the escape hole was placed in the target location. For each trial, the mouse was allowed to explore the maze freely for 180 seconds or until entering the escape hole; if the mouse did not reach the escape hole, the experimenter gently guided it there. Each apparatus was thoroughly cleaned and wiped down with 70% ethanol prior to each trial to eliminate the potential influence of olfactory cues.

The time to identify the correct (escape) hole on the final day is compared between groups. Primary latency, defined as the time for the mouse to make a first nose poke into the escape hole, was assessed. Statistical analysis was performed in GraphPadPrism 8, using ANOVA.

### Results

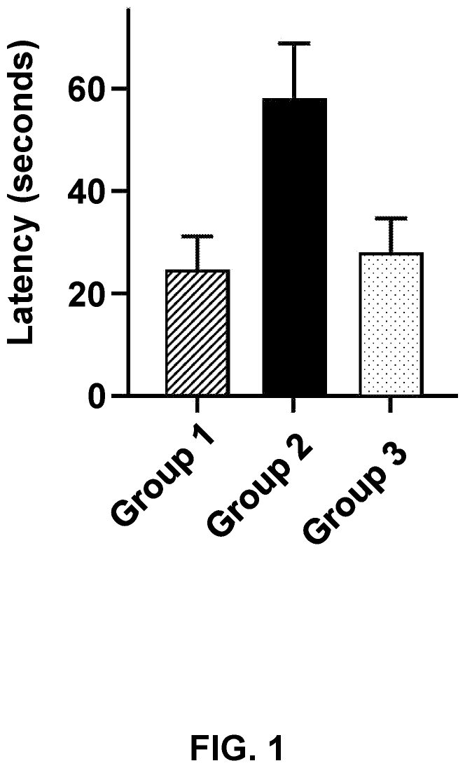

Treated and untreated APOE4 mice explored the Barnes maze during habituation at approximately the same speed. As shown in FIG. 1, on training day 4, Group 2 doxorubicin-treated mice exhibited higher primary latency (58.2 seconds) as compared to Group 1 control mice (24.6 seconds) (p===006). Group 3 doxorubicin-FPS-ZMI mice showed no delay in the latency to identify the escape hole (28.0 seconds) (p=0.12, compared to Group 2 doxorubicin-treated mice). These results suggest that RAGE antagonism prevented doxorubicin impairment of spatial learning in APOE4 mice.

### Example 2

A randomized, phase ⅔, placebo-controlled trial is conducted to assess if azeliragon (TTP488), a small-molecule RAGE inhibitor, can decrease cancer related cognitive decline in women with early stage breast cancer who are planned to undergo adjuvant or neoadjuvant chemotherapy for breast cancer treatment. The objectives and endpoints of the study are presented in Table 2 and Table 3, respectively.

TABLE 2
Objectives of the study.

| | |
|---|---|
| Primary | To assess the change in cognition of women with early breast cancer |
| | who received adjuvant or neo-adjuvant chemotherapy, randomized to |
| | azeliragon or placebo from pre-treatment to 4-6 weeks post- |
| | chemotherapy completion, prior to initiation of endocrine therapy, |
| | compared to women on placebo. Change in cognition can be |
| | determined by measurement of FACT-Cog PCI subscale, |
| | neuropsychological test of attention, processing speed, executive |
| | function, learning and memory. |
| Secondary | To assess cognitive changes from pre-treatment to 4-6 weeks post- |
| | chemotherapy completion, prior to initiation of endocrine therapy |
| | (determined by measurement of FACT-Cog PCI subscale, |
| | neuropsychological test of attention, processing speed, executive |
| | function, learning and memory) of women with non-metastatic breast |
| | cancer who received (neo)adjuvant chemotherapy, randomized to |
| | azeliragon or placebo by APOE genotype. |
| | To assess cognitive changes from pre-treatment to 12 and 24- months |
| | post-enrollment prior to initiation of endocrine therapy (determined by |
| | measurement of FACT-Cog PCI subscale, neuropsychological test of |
| | attention, processing speed, executive function, learning and memory) |
| | of women with non-metastatic breast cancer who received (neo)adjuvant |
| | chemotherapy, randomized to azeliragon or placebo. |
| | To evaluate the safety and tolerability, as measured by Common |
| | Terminology Criteria for Adverse Events (CTCAE) v. 5, of azeliragon |
| | when administered with (neo)adjuvant chemotherapy regimens for early |
| | breast cancer. |
| Exploratory | To qualify circulating cytokine, RAGE ligands, RAGE levels, and |
| | biomarkers of aging in women treated with azeliragon vs. placebo and |
| | within subgroups based on APOE genotype. |
| | To explore the impact of azeliragon vs. placebo on patient-reported |
| | outcomes (PROs) including PRO-CTCAE, and functioning as measured |
| | by the FACT-G, fatigue as measured using the FACT-fatigue, and sleep |
| | quality using the Pittsburgh Sleep Index. |
| | To explore differences in disease free survival among the treatment |
| | arms. |
| | To assess markers of metabolic syndrome including: HbA1c, Hip to |
| | waste ratio, and lipid profile. |

TABLE 3
Endpoints of the study.

| | |
|---|---|
| Primary | Less decline [defined as fewer self-reported cognitive problems (FACT- |
| | Cog PCI subscale) and scores on neuropsychological test of attention, |
| | processing speed, executive function, learning and memory] from baseline |
| | compared to 12 months after enrollment in women with early breast cancer |
| | who received (neo)adjuvant chemotherapy randomized to azeliragon vs |
| | placebo. |
| Secondary | Less decline [defined as fewer self-reported cognitive problems (FACT- |
| | Cog PCI subscale) and scores on neuropsychological test of attention, |
| | processing speed, executive function, learning and memory] from baseline |
| | compared to 12 months after enrollment in women with early breast cancer |
| | who received (neo)adjuvant chemotherapy randomized to azeliragon vs |
| | placebo. |
| | Less decline [defined as fewer self-reported cognitive problems (FACT- |
| | Cog PCI subscale) and higher scores on neuropsychological test of |
| | attention, processing speed, executive function, learning and memory] from |
| | baseline compared to 24 months after enrollment in women with early |
| | breast cancer who received (neo)adjuvant chemotherapy randomized to |
| | azeliragon vs placebo. |
| | Less decline [defined as fewer self-reported cognitive problems (FACT- |
| | Cog PCI subscale) and scores on neuropsychological test of attention, |
| | processing speed, executive function, learning and memory] from baseline |
| | compared to 4-6 weeks post-chemotherapy in women, <50 vs >50 or |
| | APOE4+ vs. negative, with early breast cancer who received (neo)adjuvant |
| | chemotherapy randomized to azeliragon vs placebo. |
| | Less decline in cognition [defined as fewer self-reported cognitive |
| | problems (FACT-Cog PCI subscale) and scores on neuropsychological test |
| | of attention, processing speed, executive function, learning and memory] |
| | from baseline to 4-6 weeks post-chemotherapy in women with APOE4 |
| | genotype, with early breast cancer who received (neo)adjuvant |
| | chemotherapy randomized to azeliragon vs placebo. |
| | Incidence and severity of adverse events, graded according to CTCAE v.5. |
| | Endpoint for Pharmacokinetics (PK). |
| Exploratory | Changes in circulating cytokines, RAGE ligands, and RAGE levels, and |
| | biomarkers of aging. |
| | Change from baseline in self-reported concentration and memory as graded |
| | by PRO-CTCAE, and functioning as measured by the FACT-G, fatigue as |
| | measures using the FACT-fatigue, and sleep quality using the Pittsburgh |
| | Sleep Index. |
| | Changes in Hip to waste ratio, Lipid profile, HbA1C. |
| | Assess difference in disease free survival in the two arms. |

### Overall Design

A total of 232 patients are block randomized by treatment site, 1:1, to either a treatment group or a placebo group. Randomization is stratified by type of chemotherapy received.

Patients randomized to the treatment group receive 15 mg of azeliragon daily (as three capsules of 5 mg each), orally with food, continuously, starting 7 days prior (day −7) to the first cycle of chemotherapy administration. At day −1, patients receive 5 mg/day of azeliragon (as one capsule of 5 mg), to continue for one year after the last cycle of chemotherapy. There are no dosage adjustments for azeliragon, even if there are chemotherapy dose reductions. If, for any reason, chemotherapy is delayed, patients continue azeliragon without dose interruptions until the end of all planned chemotherapy.

Patients randomized to the placebo group is similarly administered the placebo daily as three capsules, starting 7 days prior (day −7) to the first cycle of chemotherapy administration. At day −1, patients receive the placebo as one capsule, to continue for one year after the last cycle of chemotherapy.

End of study is defined as the point when all patients have been followed for at least 24 months after randomization.

### Participants

Eligible participants include patients with clinical or pathologic stage I-III breast cancer (including estrogen receptor (ER)+, progesterone receptor (PR)+, human epidermal growth factor receptor 2 (HER2)+, and triple negative) who are planned to receive adjuvant or neoadjuvant chemotherapy with one of three regimens: dose dense doxorubicin plus cyclophosphamide followed by paclitaxel (ddAC/ddT); docetaxel plus cyclophosphamide (TC); or docetaxel carboplatin, trastuzumab, and pertuzumab (TCHP).

### Inclusion Criteria are as Follows:

1. Patients must have clinical or pathologic stage I-III, histologically confirmed breast cancer, with any ER, PR, or HER2 status who are planned to receive chemotherapy in the adjuvant or neoadjuvant setting.
Chemotherapy regimens administered per USPI label:
   a. ddAC/ddT for 8 cycles,
   b. TC for 4-6 cycles,
   c. TCHP for 6 cycles.
2. Patients must have had no prior chemotherapy/radiotherapy/or systemic therapy for early stage breast cancer, or any other malignancy
3. Age≥18 years.
4. Women of child-bearing potential and men must agree to use adequate contraception (hormonal or barrier method of birth control; abstinence) prior to study entry and for the duration of study participation. Should a woman become pregnant or suspect she is pregnant while she or her partner is participating in this study, she should inform her treating physician immediately.
5. Ability to understand and the willingness to sign a written informed consent document.
6. Mini-Mental State Examination (MMSE) score >24 and the Wide Range Achievement Test, 4th edition (WRAT-4) Word Reading subtest score at 3rd grade or higher.

### Exclusion Criteria are as Follows:

1. Patients who have had prior chemotherapy, radiotherapy, systemic therapy, or hormonal therapy.
2. Patients with Stage IV breast cancer.
3. Patients who are receiving any other investigational agents.
4. History of allergic reactions attributed to compounds of similar chemical or biologic composition to azeliragon, docetaxel, cyclophosphamide, carboplatin, adriamycin, taxol, trastuzumab, pertuzumab.
5. Patients receiving any medications or substances that are strong CYP2C8 inhibitors are ineligible. Because the lists of these agents are constantly changing, it is important to regularly consult a frequently-updated medical reference. As part of the enrollment/informed consent procedures, the patient is counseled on the risk of interactions with other agents, and what to do if new medications need to be prescribed or if the patient is considering a new over-the-counter medicine or herbal product.
6. Uncontrolled intercurrent illness including, but not limited to, ongoing or active infection, symptomatic congestive heart failure, unstable angina pectoris, cardiac arrhythmia, neurogenerative disease/impairment, or psychiatric illness/social situations that would limit compliance with study requirements.
7. Pregnant women are excluded from this study; breastfeeding should be discontinued if the mother is treated with azeliragon. These potential risks may also apply to other agents used in this study.
8. History of cancer within the last 5 years except adequately treated cervical carcinoma-in-situ, cutaneous basal cell, or squamous cell cancer

### Concomitant Therapy

All prior treatment or medication administered during the 30 days prior to enrollment and any concomitant therapy administered to the patient throughout the study until date of surgery must be recorded.

Dose dense doxorubicin, cyclophosphamide, paclitaxel, docetaxel, carboplatin, trastuzumab, and pertuzumab is administered per standard operating procedures at each institution.

Regarding other concomitant therapy, prophylactic granulocyte colony stimulating factor (G-CSF) is administered per institutional guidelines. Patients should receive best supportive care and treatment of symptoms during the study as appropriate, including treatment with appropriate medications (antibiotics, antiemetics, antidiarrheals, and analgesics, etc.).

### Study Assessments

During the Screening Phase, i.e., the time between the date a patient provides written informed consent, and the time the patient completes enrollment procedures/has enrollment verified, data is collected that may include patient demographics, eligibility requirements, concomitant medications, medical history, physical examination/vital signs, ECOG performance status assessment, adverse events, serious adverse events, laboratory measurements, pregnancy testing (if applicable), and biospecimens (blood and tissue samples). All laboratory assessments must be performed within 28 days of treatment start.

During the Treatment Phase, i.e., the period in which the patients are receiving treatment (azeliragon or placebo), patients may undergo clinical evaluation/medical history/physical examinations, vital sign determinations, performance evaluations, clinical laboratory tests (e.g., complete blood count, comprehensive metabolic panel), and neuropsychological assessment.

The neuropsychological assessment includes FACT-Cog PCI subscale and tests for attention, processing speed, executive functioning, learning, and memory. Participants with an MMSE score >24 and a WRAT-4 score >3rd grade reading proceed to neuropsychological testing.

Tests for evaluating executive functioning, working memory, and psychomotor speed may include Oral Trailmaking—Part A; Oral Trailmaking—Part B.; Digit Symbol Subtest-Wechsler Adult Intelligence Test-III; Symbol Digit Modalities Test; COWAT; Mental Control Subtest-Weschler Memory Scale; Driving Scenes Test from the NAB; TIADL; and Figure Drawing from the NAB—Organization Subscale. Tests for evaluating language may include BNT; Verbal Naming; and Category Fluency Test. Test for evaluating attention may include NAB Digits Forward; NAB Digits Backward; and Brief Test of Attention. Tests for evaluating learning and memory may include Logical Memory I and II, Wechsler Memory Scale (WMS-IV); and List Learning from NAB. Finally, test for evaluating visual-spatial functioning may be the Figure Drawing from NAB.

Quality of life is assessed using Patient-Reported Outcomes Common Terminology Criteria for Adverse Events (PRO-CTCAE) measurement system, FACT Fatigue, FACT G and B (functioning), and Pittsburgh Sleep Index to evaluate sleep disturbances.

Blood samples may be drawn to measure circulating cytokines, RAGE ligands, RAGE levels, sRAGE, and APOE4, as well as biomarkers of aging, DNA repair, and cellular senescence.

In addition, adverse events are assessed throughout the study, from the time Of administration of the treatment until 60 days after discontinuation of the therapy.

The foregoing description is given for clearness of understanding only, and no unnecessary limitations should be understood therefrom, as modifications within the scope of the invention may be apparent to those having ordinary skill in the art.

Detailed embodiments of the present methods and compositions are disclosed herein; however, it is to be understood that the disclosed embodiments are merely illustrative and that the methods and compositions may be embodied in various forms. In addition, each of the examples given in connection with the various embodiments of the systems and methods are intended to be illustrative, and not restrictive.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

### REFERENCES

Ahles T A, et al. The relationship of APOE genotype to neuropsychological performance in long-term cancer survivors treated with standard dose chemotherapy. Psychooncology 12: 612-619 (2003).
Ahles T A and Saykin A J. Candidate mechanisms for chemotherapy-induced cognitive changes. Nature Reviews Cancer 7: 192-201 (2007).
Ahles T A, et al. Cancer- and cancer treatment-associated cognitive change: an update on the state of the science. Journal of Clinical Oncology 30: 3675-3686 (2012).
Anderson-Hanley C, et al. Neuropsychological effects of treatments for adults with cancer: A meta-analysis and review of the literature. Journal of the International Neuropsychological Society 9: 967-982 (2003).
Benton A L. Neuropsychological assessment. Annual Review of Psychology 45: 1-23 (1994).
Bluethmann S M, et al. Cognitive function and discontinuation of adjuvant hormonal therapy in older breast cancer survivors: CALGB 369901 (Alliance). Breast Cancer Research and Treatment 165: 677-686 (2017).
Brown L B, et al. Driving scenes test of the Neuropsychological Assessment Battery (NAB) and on-road driving performance in aging and very mild dementia. Archives of Clinical Neuropsychology 20: 209-215 (2005).
Deane R, et al. A multimodal RAGE-specific inhibitor reduces amyloid β-mediated brain disorder in a mouse model of Alzheimer disease. Journal of Clinical Investigation 122: 1377-1392 (2012).
Demby T C, et al. A mouse model of chemotherapy-related cognitive impairments integrating the risk factors of aging and APOE4 genotype. Behavioural Brain Research 384: 112534 (2020).
Han Y T, et al. Ligand-based design, synthesis, and biological evaluation of 2-aminopyrimidines, a novel series of receptor for advanced glycation end products (RAGE) inhibitors. Journal of Medicinal Chemistry 55: 9120-9135 (2012).
Han Y T, et al. Pyrazole-5-carboxamides, novel inhibitors of receptor for advanced glycation end products (RAGE). European Journal of Medicinal Chemistry 79: 128-142 (2014).
Han Y T, et al. Fine tuning of 4,6-bisphenyl-2-(3-alkoxyanilino)pyrimidine focusing on the activity-sensitive aminoalkoxy moiety for a therapeutically useful inhibitor of receptor for advanced glycation end products (RAGE). Bioorganic & Medicinal Chemistry 23: 579-587 (2015).
Hudson B I and Lippman M E. Targeting RAGE signaling in inflammatory disease. Annual Review of Medicine 69: 349-364 (2018).
Janelsins, M C, et al. Prevalence, mechanisms, and management of cancer-related cognitive impairment. International Review of Psychiatry (Abingdon, England) 26: 102-113 (2014).
Kalea A Z, et al. RAGE: a novel biological and genetic marker for vascular disease. Clinical Science (London) 116: 621-637 (2009).
Kaplan E, et al. The Boston naming test. Philadelphia, PA (1983).
Kierdorf K and Fritz G. RAGE regulation and signaling in inflammation and beyond. Journal of Leukocyte Biology 94: 55-68 (2013).
Lange M, et al. Cognitive dysfunctions in elderly cancer patients: a new challenge for oncologists. Cancer Treatment Reviews 40: 810-817 (2014).
Lee Y S, et al. Synthesis and structure-activity relationships of tri-substituted thiazoles as RAGE antagonists for the treatment of Alzheimer's disease. Bioorganic & Medicinal Chemistry Letters 22: 7555-7561 (2012).
Lezak M D, et al. Neuropsychological Assessment Fourth Edition. New York, NY, Oxford University Press (2004).
Mandelblatt J S, et al. Cognitive effects of cancer systemic therapy: implications for the care of older patients and survivors. Journal of Clinical Oncology 32: 2617-2626 (2014).
Mandelblatt J S, et al. Cancer-related cognitive outcomes among older breast cancer survivors in the thinking and living with cancer study. Journal of Clinical Oncology. 36: 3211-3222 (2018).
Neeper M, et al. Cloning and expression of a cell surface receptor for advanced glycosylation end products of proteins. Journal of Biological Chemistry 267: 14998-15004 (1992).
Owsley C, et al. Timed instrumental activities of daily living tasks: relationship to cognitive function and everyday performance assessments in older adults. Gerontology 48: 254-265 (2002).
Speidell A P, et al. Development of a human APOE knock-in mouse model for study of cognitive function after cancer chemotherapy. Neurotoxicity Research 35: 291-303 (2019).

What is claimed is:

1. A method of treating cancer-related cognitive decline (CRCD) in a patient in need thereof, the method comprising administering to the patient an effective amount of an inhibitor of receptor for advanced glycation endproducts (RAGE).

2. A method of preventing cancer-related cognitive decline (CRCD) in a patient in need thereof, the method comprising administering to the patient an effective amount of an inhibitor of receptor for advanced glycation endproducts (RAGE).

3. A method of alleviating symptoms of cancer-related cognitive decline (CRCD) in a patient in need thereof, the method comprising administering to the patient an effective amount of an inhibitor of receptor for advanced glycation endproducts (RAGE).

4. A pharmaceutical composition comprising an effective amount of an inhibitor of receptor for advanced glycation endproducts (RAGE) for use in treating cancer-related cognitive decline (CRCD) in a patient in need thereof.

5. A pharmaceutical composition comprising an effective amount of an inhibitor of receptor for advanced glycation endproducts (RAGE) for use in preventing cancer-related cognitive decline (CRCD) in a patient in need thereof.

6. A pharmaceutical composition comprising an effective amount of an inhibitor of receptor for advanced glycation endproducts (RAGE) for use in alleviating symptoms of cancer-related cognitive decline (CRCD) in a patient in need thereof.

7. The method or composition of any preceding claim 7, wherein the CRCD is induced by a hormonal cancer therapy or a chemotherapy.

8. The method or composition of claim 7, wherein the CRCD is induced by a chemotherapy.

9. The method or composition of any preceding claim, wherein the patient comprises an apoliprotein E4 genotype.

10. The method or composition of any preceding claim, wherein the inhibitor of RAGE is administered as a single dose before the administration of the cancer therapy, during the administration of the cancer therapy, or after the administration of the cancer therapy.

11. The method or composition of any one of claims 1-9, wherein the inhibitor of RAGE is administered as multiple doses for a duration of time.

12. The method of composition of claim 11, wherein the duration of time is before the administration of the cancer therapy, during the administration of the cancer therapy, after the administration of the cancer therapy, or a combination thereof.

13. The method of claim 1 or the pharmaceutical composition of claim 3, wherein the administration of the inhibitor of RAGE improves attention, processing speed, executive functioning, learning, memory, visuospatial ability, or a combination thereof, of the patient as compared to patients who are not administered the inhibitor of RAGE.

14. The method of claim 2 or the pharmaceutical composition of claim 4, wherein the administration of the inhibitor of RAGE prevents reduction in attention, processing speed, executive functioning, learning, memory, visuospatial ability, or a combination thereof, of the patient.

15. The method of claim 3 or the pharmaceutical composition of claim 6, wherein the administration of the inhibitor of RAGE alleviates symptoms of CRCD demonstrated by a reduction in attention, processing speed, executive functioning, learning, memory, visuospatial ability, or a combination thereof, of the patient.
